(19) United States
(12) Patent Application Publication
(10) Pub. No.: US 2005/0220665 A1
(43) Pub. Date: Oct. 6, 2005

(54) Low temperature sterilization and disinfections method and apparatus for medical apparatus and instruments

(76) Inventor: Lambert Lishing Ding, Cerritos, CA (US)

Correspondence Address: PHILIP K. YU, 20955 PATHFINDER ROAD, SUITE 100, DIAMOND BAR, CA, 91765, US

(21) Appl. No.: 10/952,093

(22) Filed: Sep. 28, 2004

Related U.S. Application Data
(60) Provisional application No. 60/559,506, filed on Apr. 5, 2004.

(51) Int. Cl. A61L 2/18; A61L 2/025; A61L 2/10

(52) U.S. Cl. 422/20; 422/24; 422/28; 422/128; 422/300

(57) ABSTRACT

A method and apparatus for the low temperature sterilization and disinfections to be applied to medical apparatus/instruments. This invention perfects the use of ultrasonic, UV, ozone in a liquid bath with at least one chemical reagent which can be either sterilant or disinfectant solution for reprocessing many classes of medical equipment/instruments especially those which do not withstand high temperature sterilization process and require sterilization or high level disinfections. The invented method can effectively remove and destroy contaminates, particularly living organisms, bacterial spores, virus, and organics from used or contaminated medical apparatus/instruments surface, inner hollow lumen tube, joints and crevices. Having greatest immediate application for the cleaning, disinfecting and sterilizing all in one simple process for reprocessing used or contaminated medical apparatus/instruments after treating patients.

### RELATED APPLICATION

The instant application claims priority from a provisional application filed on Apr. 5, 2004, Ser. No. 60/559,506, entitled LOW TEMPERATURE STERILIZATION AND DISINFECTIONS METHOD FOR MEDICAL APPARATUS/INSTRUMENTS, by Applicant, the disclosure of which is incorporated herein by reference.

### BACKGROUND OF THE INVENTION

1. Field of Invention

The invention generally relates to the field of reprocessing medical equipment. In particular it relates to an improved reprocessing device and method for sterilizing, as well as cleaning and disinfecting for used or contaminated medical apparatus/instruments such as endoscopes to be used in medical, dental, heart surgery and operations, and kidney dialysis instruments and all medical equipment that can be subject to low temperature disinfections or sterilization, by applying ultrasonic vibration along with ultraviolet (UV) irradiation and ozone into the liquid bath containing disinfectants to completely remove and destroy adhered microorganisms contaminates from within and above. This invention relates to, in particular, the medical apparatus/instruments which do not withstand high temperature sterilization process, and can be cleaned, disinfected and sterilized all in one single efficient process.

2. Brief Description of the Prior Art

It has been quite common in the medical field to re-process used medical apparatus and instruments, due to the cost of such medical apparatus and instruments. The problem often occurs in conventional medical practices of reprocessing used medical apparatus/instruments with bacterial spores adsorbed to the complicated designed medical lumen—predominately but not limited to endoscopes.

The apparatus/instruments that are used to treat patients become contaminated when organic contaminates such as body fluid, blood and bacterial spores are adsorbed and hardened on the surfaces, interior lumen tubing and porous joints of the apparatus/instruments. Such absorption is a physical phenomenon in that organic compounds are attracted by van der Waals forces and become physically attached to the surfaces of the medical apparatus/instruments. Government standards require these apparatus/instruments to be reprocessed either by sterilization or high-level disinfections to prevent cross infection of diseases to the next treated patient. However, the US Food and Drug Administration (FDA) requirements of cleaning and sterilization process with combination of manual cleaning and machine cleaning are tedious and time consuming. Even so, due to the complicated design of contemporary endoscopes, the lumen hollow tubing and joints are hard to clean and cross infections are often observed and sometimes results in deaths and legal disputes.

Concerns of surgeons and health professionals about cross infection in general emphasize that effective cleaning and decontamination of endoscopes is vital. Flexible endoscopes have fragile parts, are easily damaged at high temperatures and are very expensive. So they cannot be autoclaved and instead must be decontaminated by cleaning at low temperatures and disinfections. Not only has there been a vast increase in this type of minimal invasive surgery but there is far more awareness of the dangers of cross infection by hospitals and the public. A major problem with conventional endoscopes washer-disinfectors is that they simply sit and soak endoscopes in the disinfectants or sterilant, which does not ensure the removal nor destruction of all living organisms adhered thereto.

Currently, Olympus washer-disinfector is the most popular device to be used in medical fields for reprocessing of endoscopes; it is designed to flush the contaminated endoscopes with Cidex, a sterilizing agent to achieve high-level disinfections purpose. However, cross infection and medical dispute cases are often heard still, due to many studies showing that the simple flushing technique does not ensure the complete removal and destruction of complicated bacterial spores. Often the outer sphere of the living organisms are destroyed by contact of sterilant, while the inner lumen of the endoscopes with attached bacterial spores create a barrier to protect itself from further reacting with the sterilant. And after the process is considered complete with the adverse sterilant environment being released, the un-removed bacterial spores can come back to active and infect the next patient. That's why the government and Society of Endoscopes are eager to see a new technology to be developed to deal with these cross-infection issues that have become more and more frequently encountered these days.

Disposal of contaminated medical apparatus/instruments would be the best practice to prevent cross contamination problems; however, the high cost and complicated design of many medical apparatus/instruments have prevented this effective option. The current existing technology commonly known as low temperature disinfections or sterilization can be described as follows:
A: Sterilization: done by immersing medical apparatus/instruments into a system with sterilant and circulating the liquid bath through the apparatus/instruments for a certain period of time to achieve the required sterilization.
B: High-Level Disinfections: done by immersing medical apparatus/instruments into a system with disinfectants solution such as Cidex, glutaraldehyde, and circulating the liquid bath through the apparatus/instruments for a certain period of time to achieve the FDA required high-level disinfections.

However, the above mentioned existing flushing technologies along with sterilant or disinfectants do not provide adequate desorbing mechanisms for contaminates and microorganisms attached to the complicated lumen tubing joints and crevices, and often result in cross-infection and even deaths.

Most medical equipment can be sterilized at high temperatures. Commonly, the equipment is sterilized in an autoclave under a combination of high temperature and pressure steam. While such sterilization methods are very effective for more resilient medical apparatus/instruments, more sensitive medical apparatus/instruments made of rubber and plastic materials with adhesives will be damaged by the high temperature and pressure steam autoclave. In particular, the costly complex instruments such as endoscopes may be destroyed or have their useful lives greatly shortened by high temperature and pressure thermal sterilization methods. Further, endoscopes present particular problems in that such devices typically have numerous exterior crevices and interior lumens that can retain microbes and hence difficult to clean and sterilize using conventional flushing techniques along with disinfectants or sterilant such as Cidex, Glutaraldehyde, Hydrogen peroxide, Alcohols, Ethylene oxide, Formaldehyde, and Peracetic acids etc. Therefore, the need for a fast acting, simple and effective decontamination process to achieve sterilization and disinfections is more desirable for reprocessing sensitive instruments, such as endoscopes.

So far, efforts to sterilize more sensitive medical instruments, such as endoscopes, have met with limited success and all conventional methods have associated problems or detractions. Sensitive medical instruments, such as endoscopes, are usually sterilized with above-mentioned flushing techniques, and often with ethylene oxide method, which is thermally less severe than steam. The endoscopes must be exposed to the ethylene oxide for a relatively long period of time, on the order of three and a half hours. Thereafter, eight to twelve hours are normally required for de-gassing or desorbing the ethylene oxide from plastic and other materials that are capable of absorbing the ethylene oxide. The pressurization and depressurization cycles of ethylene oxide sterilization may damage lens systems and other delicate instruments that are commonly integral with endoscopes. Moreover, the ethylene oxide is relatively expensive. It is sufficiently toxic and volatile that extensive precautions are commonly taken to assure operator safety.

Liquid systems are commonly used for disinfecting endoscopes and other heat sensitive and delicate instruments. Using liquid sterilant or disinfectants to achieve disinfections is normally rapid, cost-effective and does minimal damage to the medical devices. Conventionally, a technician mixes a sterilant composition and manually immerses the item to be disinfected. Technician variation in the mixing, timing, and equipment handling raises problems of assurance and reproducibility of the manual disinfections process. Rinsing of the items to remove chemical residues also adds a variable that reduces the assurance of disinfections or sterility. Once, rinsed, the disinfected endoscopes or other item is susceptible to recontamination by airborne microbes. Further, merely soaking endoscopes in a sterilant or disinfectant is unacceptable since numerous pockets exist within the tubing that the sterilant or detergent cannot reach effectively. This leaves areas of contamination within the endoscopes. With the prevalence of highly contagious diseases such as Hepatitis B and Acquired Immune Deficiency Syndrome, effective sterilization, or disposal, of all medical tools becomes mandatory. Accordingly, an ineffective effort to sterilize endoscopes by merely soaking is unacceptable.

For example, U.S. Pat. No. 5,091,343 discloses a liquid sterilization system involving placing the instrument to be sterilized in a tray or cassette that is then covered and positioned within a liquid sterilization unit. A major drawback of this type of process is the lack of assurance of a sufficient flow of sterilant and rinse water through the interior passages of the instrument. The simple circulation of the liquid sterilant in the cassette or tray and the numerous pockets inherent in such a tubular instrument provides no assurance that adequate sterilization is attained in the interior passages of the instrument. The exterior surfaces of instruments, such as endoscopes, typically have multiple connectors and branches that can define small crevices or niches harboring microbes. Because of this, the circulation liquid sterilization systems, which rely on complete submersion of the endoscopes, may also be inadequate to assure complete sterilization of all exterior surfaces. U.S. Pat. No. 6,585,934 presents a reprocessing system including a reaction chamber into which individual chemical components of a sterilant are transferred by pneumatic force, mainly circulating sterilant to achieve sterilization purpose.

Sterilization by immersing the medical devices in an atmosphere that is antagonistic to the survival of the microbiological forms include, but is not limited to, steam, alcohols, ethylene oxide, formaldehyde, glutaraldehyde, hydrogen peroxide, and peracids. Each of these materials has its benefits and limitations. Ethylene oxide tends to be very effective against a wide range of microorganisms, but it is highly flammable, longer process time as mentioned previously and is generally used in a gas phase which may require more stringent environmental restraints than would a liquid. Alcohols are similarly flammable and must be used in very high concentrations. Steam has a more limited utility, having to be used in a controlled and enclosed environment, requiring the use of large amounts of energy to vaporize the water, and requiring prolonged exposure periods to assure extended high temperature contact of the steam with the organisms. Hydrogen peroxide has limited applicability because it is unstable and not as strong as some other sterilant. The peracids have become more favorably looked upon, but they tend to be in high concentration, corrosive (being an oxidizing acid) and are not shelf stable.

U.S. Pat. No. 5,508,046 describes a stable, anticorrosive peracetic acid/peroxide sterilant comprising a concentrate including peracetic acid, acetic acid, hydrogen peroxide and stabilizers such as phosphoric acids and sodium pyrophosphates. However, the flushing of sterilant presents the same associated problem encountered due to interior air pockets and lack of quality assurance for completely removal of the interior lumen microorganisms.

U.S. Pat. No. 4,892,706 describes automated liquid sterilization systems having a plurality of modules which store the sterilant solution and the rinse solution. U.S. Pat. No. 5.037,623 describes a sterilant concentrate injection system which is a spill resistant, vented ampule system for use with sterilization systems.

Sterilization can destroy all forms of life, including bacterial spores, the living organisms most resistant to sterilant. Microbial decontamination refers to both sterilization and disinfections. If a reprocessing method can ensure the medical apparatus/instruments be free of living organisms not only from the surface but also from within the lumen hollow tube, and ensure all the removed living organisms are killed and destroyed after the process, then it can achieve the sterilization goal and prevent cross contamination from occurring. A new and efficient method is invented for low temperature sterilization and disinfections of the contaminated medical apparatus/instruments with lumens, such as endoscopes.

### SUMMARY OF THE INVENTION

The invented method and apparatus are to overcome the drawback of the known sterilization and disinfections practices and to provide a method that can ensure the quality of cleaning, disinfections and sterilization of all the medical apparatus/instruments, especially but not limited to endoscopes.

The invented method and apparatus described herein are a low temperature sterilization and disinfections process. The invention perfects the use of ultrasonic, UV, and ozone in a liquid sterilant or disinfectant solution. The invented method and apparatus can be applied to many classes of medical equipment/instruments, especially but not limited to those which do not withstand high temperature sterilization process.

The present invention is also directed to a process for rendering bacterial endospores harmless and lifeless, where the method comprising the following steps: (a) including means for desorbing attached living organisms contaminates from the medical equipment/instruments by ultrasound cavitations forces; and (b) adding an effective amount of sterilant/disinfectants agent into the liquid bath and re-circulating in and out of the endoscopes; and (c) applying ozone into the liquid stream; (d) applying UV light sources to enhance the ozone reaction and create a bacterial free environment.

The invention is also directed to a method of killing spores comprising the steps of: (a) combining in an aqueous solution an effective amount of sterilizing or disinfecting agents for said spores; and (b) contacting said spores with said aqueous solution therefore killing said spores; (c) providing ozone, a strong oxidizing agent that can destroy and decompose living organics upon contact both in liquid phase as well as in gas phase to deal with potential air pockets within the lumen tubing; (d) applying UV, an effective and economical energy source for killing bacteria.

This invented method and apparatus can be achieved by means of a device for cleaning, disinfecting and sterilizing medical apparatus/instruments, especially but not limited to endoscopes that may have a plurality of channels, comprising at least one rinsing basin, in which endoscopes to be sterilized or disinfected can be placed, a housing in which, a system of lines, pumps and valves is accommodated for the purpose of feeding a combination of sterilizing or disinfecting agents to endoscopes which has been placed in a rinsing basin, means for connecting the channels of an endoscope which has been placed and immersed in the basin to the system of lines, pumps and valves, and control means for controlling the ultrasound generator, UV power, ozone generator, pumps and valves, etc. A system with ultrasound generators or probes to be installed to the reactor basin, and UV light sources equipped to the top or within the reactor, and ozone can be introduced by passing oxygen or air through the ozone generator and via ozone distributor to be purged into the basin to deliver saturated ozonated stream for reaction.

The device and method according to the invention allow the endoscopes to be ultrasonically desorbed, sterilizing agents flushed, along with UV and ozone decontamination reactions, so that the sterilization and disinfections is more efficient and operationally reliable than the known systems. The system according to the invention furthermore has the additional advantage that the process can be carried out at room temperature, so that the adverse effects of sterilizing and disinfecting at elevated temperature (for example above 80.degree. C.) are eliminated.

### OBJECTS OF THE INVENTION

It is an object of this invention to provide a faster and more effective method for decontamination in cleaning and sterilization as well as disinfections for medical apparatus/instruments and implants.

It is also an object of this invention to provide a low temperature sterilization method for cleaning, disinfecting and sterilizing medical apparatus/instruments and implants.

It is also an object of this invention to provide an alternative to high temperature sterilization for sterilizing or disinfecting medical apparatus/instruments and implants.

It is an additional object of this invention to provide sterilization and disinfections method for most medical apparatus/instruments and implants, especially but not limited to those which do not withstand high temperatures or pressures.

It is another object of the present invention to provide an improved device for fast reprocessing and sterilizing medical instruments with lumens, such as endoscopes in combination with one or more sterilant to sterilize objects containing microorganisms, such as bacterial spores.

It is another object of the present invention to provide an improved method for sterilizing medical instruments with lumens, such as endoscopes with potential air pockets problems, with ozone being trapped in the air pockets of the lumen, it is effective both in liquid and gas phases for decontaminating and sterilizing microorganisms.

### BRIEF DESCRIPTION OF THE INVENTION

This invention is directed to a method and apparatus for the cleaning, disinfections and sterilization of contaminates, particularly living organisms from used or contaminated medical apparatus/instruments.

In the method of this invention, the used or contaminated medical apparatus/instruments is immersed in an aqueous bath, preferably a sterilant/disinfectant or combination of various sterilansts/disinfectants agent can be added to the aqueous bath at a concentration from 1 to 200,000 ppm, and the aqueous bath is circulated over the used or contaminated medical apparatus/instruments while ultrasonic vibration is applied to the aqueous bath at a frequency from about 1 to about 100 MHz, sufficient to effect a substantial desorption and removal of the living organisms contaminates from the medical apparatus/instruments. The attached living organisms contaminate can be fast and effectively desorbed and removed by ultrasound cavitations forces and washed away from the medical apparatus/instruments, and then killed by the circulating sterilant/disinfectant liquid stream. Preferably the desorbed living organisms contaminates are destroyed by introducing the strong oxidizing agent ozone into the liquid stream, and the saturated ozonated stream can be gasified by the ultrasonic cavitations and trapped in the potential air pockets of the lumen tubing of the medical apparatus/instruments as well as the gas phase of the reactor chamber to conduct oxidation and destruction of the microorganisms, most preferably, while subjecting the ozonated aqueous stream to UV irradiation, with the UV wavelength range from 10 to 400 nm, thereby effecting and enhancing the UV/ozone catalytic destruction of the living organisms contaminates, and creating a bacterial free environment in both liquid and gas phases of the reactor chamber to ensure the decontamination purpose and quality thus achieving the sterilization and disinfections goals.

### BRIEF DESCRIPTION OF THE DRAWINGS

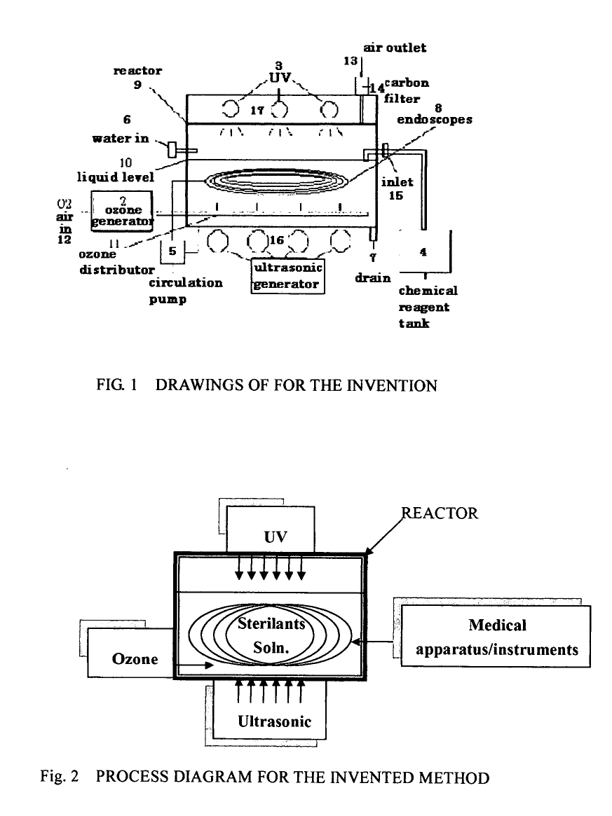

The invention will be described with reference to FIGS. 1 and 2, which are detailed drawings and flow diagrams for the process of the invention.

### DESCRIPTION OF THE PREFERRED EMBODIMENTS

This invention is directed to a method and apparatus for cleaning, disinfecting and sterilizing used and contaminated medical apparatus/instruments which contain adsorbed living organisms. The medical apparatus/instruments are prone to contamination after being used in treatment of patients who contain disease-causing microorganism contaminants such as body fluid, blood, bacterial spores, virus, etc. The microorganism contaminants are adsorbed on the surfaces of the medical apparatus/instruments by physical, van der Waals forces. In most applications the medical apparatus becomes contaminated when microorganisms have been adsorbed thereon, and the presence of the microorganism contaminates need to be removed and destroyed before the same apparatus can be used to treat the next patient.

Although specific embodiments of the present invention is described with reference to the drawings, it should be understood that such embodiments are merely illustrative of but a small number of the many possible specific embodiments which can represent applications of the teaching of the present invention. Various changes and modifications obvious to one skilled in the art to which the present invention pertains are deemed to be within the spirit, scope and contemplation of the present invention as further defined in the appended claims.

In accordance with the present invention, the method begins with immersing the medical apparatus/instruments into an aqueous liquid bath with one or more sterilant, applying ultrasonic vibration to the aqueous bath reactor chamber containing the medical apparatus/instruments. Then, the method involves supplying ozone into the reactor bath and circulating the bath to fully contact with the medical apparatus/instruments in order to oxidize and decontaminate the adsorbed living organism contaminates, with UV radiation installed at the vapor phase or in the liquid to generate photo-catalytic reaction with ozone to enhance the reaction, and create a bacterial free environment to reduce the chance of cross contamination from atmosphere.

FIG. 1 is a schematic representation of one embodiment of the apparatus of the invented method employing the principle of ultrasonic, UV, ozone in the sterilant solution bath reactor.

FIG. 2 is a schematic representation of the process of the invented method employing the principle of ultrasonic, UV, ozone in the sterilant solution bath reactor.

In FIG. 1, the water is introduced through a top water inlet nozzle 6 into the reactor 9 and is removed from the lower extremity of the reactor through drain water outlet 7. An ultrasonic probe 16, which can be a tubular member, is preferably centrally located under the reactor 9. Ultrasonic vibration is applied to this probe 16 using a conventional ultrasonic generator 1 which has a sufficient power rating for the application of ultrasonic vibrations to the medical apparatus/instruments such as endoscopes 8. The ultrasonic vibration is applied at a frequency in the range from 1 to about 100 MHz. When sterilization or disinfections is required, a medical apparatus/instruments such as endoscopes 8 will be placed and immersed beneath the liquid level 10 within the reactor 9 to be processed by this method of invention. The water is introduced through water inlet 6 with chemical reagent such as disinfectants solution pumped from chemical reagent tank 4 through inlet nozzle 15 into the reactor 9 at a predetermined concentration typically from 1 ppm to 200,000 ppm depends on various needs.

The treatment process is conducted at ambient to slightly elevated temperatures, e.g., from 20 degree C. to about 100 degree C. The water will be heated by the application of the ultrasonic vibration, and this heating will be sufficient to maintain the aforementioned temperature.

A source of oxygen is also introduced into the reactor through an oxygen and air inlet nozzle 12. Preferably the oxygen source is air from the ambience. The oxygen containing gas, either air or a mixture of oxygen with an inert gas, is passed over an ozone generator 2, which, again, is a conventional generator to which electrical energy is supplied in sufficient quantities to generate ozone in the air stream, typically providing a concentration of ozone in the air stream between about 0.1 and 5 percent. The air stream is introduced into the reactor 9 and percolated through the ozone distributors 11 into the liquid bath within the reactor at a rate sufficient to provide a concentration of ozone within the reactor of about approximately 0.5 to 2 weight percent. Excess treating gas, i.e., excess air, is removed from the reactor through carbon filter 14 to air outlet nozzle 13 and exhausted to the atmosphere.

Preferably ultraviolet irradiation is applied to the reactor. This can be achieved by locating one or more ultraviolet light sources 3 within the reactor 9. In the illustrated embodiment, an ultraviolet light source 3 is shown in the vapor space on top of the reactor 9. Alternatively the ultraviolet light source 3 could be immersed beneath the liquid level within the reactor 9. The light source 3 can have an emission wavelength in the near ultra-violet and the ultra-violet range, e.g., from 10 to about 400 nm.

The sterilization and disinfections process will be maintained for a sufficient length of time to remove and kill the living organism contaminates from the medical apparatus/instruments 8 and restore to substantially sterilized status. Typically this requires a treatment time from about 10 to 30 minutes, depending on the volume and concentration of the living organism contaminates on the apparatus/instruments.

In the illustration of FIG. 1, the medical apparatus/instruments such as endoscopes 8 is immersed in the reactor 9 and being treated in accordance with the invention. Water is introduced into the reactor 9 through water inlet 6 until it reaches a pre-set liquid level. The disinfectant in tank 4 is pumped into the reactor 9 through inlet nozzle 15. The concentration is typically from 1 ppm to 200,000 ppm, depending on various needs, and the predetermined concentration should be sufficient for living organism contaminates to be killed and destroyed upon contact with the disinfectant solution.

The circulation pump 5 withdraws the disinfectant solution from the reactor 9 and pumps into the endoscopes 8 to flush, kill and remove the ultrasound desorbed inner lumen contaminates. Vibration is supplied from the ultrasonic generator 1 to the ultrasonic probes 16 within the reactor 9. Ultraviolet radiation is supplied by source 3 on top of reactor 9. Oxygen/air mixture is passed thorough air inlet 12 to the ozone generator 2 and introduced into reactor 9 through the ozone distributors 11. Excess air products are vented from reactor 9 through carbon filter 14 to air outlet 13. After the decontamination treatment of the medical apparatus/instruments 8 in reactor 9, it will be completely cleaned, disinfected and sterilized. When the medical apparatus/instruments such as endoscopes 8 is used or contaminated again, it can be placed into the aqueous bath in reactor 9 to repeat the process in accordance with the present invention to achieve the sterilization results.

The following example will illustrate the practice of the invention and demonstrate results obtainable thereby.

### EXAMPLE

A laboratory investigation of the invented method has been conducted. Used and contaminated medical endoscopes are placed and immersed in the reactor. Clean tap water is introduced into this 12-liter reactor and filled to the pre-set liquid level. It is then spiked with bacterial samples of e-coli and yeasts with a concentration measured up to 2,000,000/liter in the reactor and contacting the endoscopes therein with prepared water bath. The contaminated medical endoscopes along with spiked water bath are homogeneously contacted and circulated to simulate the worst contaminated condition.

The ultrasonic, UV and ozone forces are then applied to the reactor. At the same time the disinfectants solution is pumped into the reactor to react with the contaminated medical endoscopes with the reaction set for a period of time (timer set at first 5 minutes). When the time is up, a drain operation is activated and followed by second sequence of clean water input, repeating the process of injecting disinfectants solution, ultrasonic vibration, UV and ozone forces for another period of time (set for second 5 minutes). When the second period expires, a drain operation is activated, and followed by third sequence of clean water input, then repeating the process of injecting disinfectants solution, ultrasonic vibration, UV and ozone forces for another period of time (set for third 5 minutes). And then the reactor bath is drained, followed by 2 clean water rinsing cycles, along with ultrasonic, UV and ozone only for a short time period (timer set for 2 minutes each), with draining after each rinsing. This will ensure no residue of the disinfectants and living organisms contaminates is left, both on the endoscopes and within the internal lumen tubing surfaces and joints.

The progress and performances of the sterilization for the endoscopes is monitored by withdrawing samples of the contaminated water and analyzing the microorganism's numbers from sample specimens using laboratory technique to detect when the water bath shows no further trace of the living organisms contaminants.

The processed endoscopes is then removed and sampled from top and inner surface with sample specimen, and sent to lab for analysis.

The first sample is taken directly from the water bath when the water bath is spiked and homogenized with E-coli and yeasts to determine the concentration of initial stage with no reaction applied to the endoscopes;

The second sample is taken at the end of the first 5 minute reaction with sample taken directly from the water bath before it is drained;

The third sample is taken at the end of second 5 minutes reaction with sample taken directly from the water bath before it is drained;

The fourth sample is taken at the end of third 5 minutes reaction with sample taken directly from the water bath before it is drained;

The fifth sample is taken at the end of first rinsing operation with sample taken directly from the water bath before it is drained;

The sixth sample is taken at the end of second rinsing operation with sample taken directly from the water bath before it is drained;

The seventh sample is taken at the end of third rinsing operation. Sample is taken by using bacterial free cotton swab and scrubbing from the outer and inner surfaces of the endoscopes directly after the endoscopes is removed from the reactor chamber. The following results are obtained:

TABLE
Process performance data

| | | Microorganisms | |
|---|---|---|---|
| Sample No. | Time | (No./liter) | Specimen |
| 1 | 0 | 2,000,000 | Solution |
| 2 | 5 | 100,000 | Solution |
| 3 | 10 | 1,000 | Solution |
| 4. | 15 | N/D | Solution |
| 5. | 17 | N/D | Solution |
| 6. | 19 | N/D | Solution |
| 7. | 19 | N/D | Cotton Swab |

Note:
1 Reactor solution volume is 12 liters, with bacterial microorganisms spiked into the solution.
2 N/D: Non-detected.

From a comparison of the total quantity of living microorganisms contaminates in the liquid bath and on the surface of the endoscopes, it is observed that the endoscopes which was sterilized with ultrasonic vibration, ozone and ultraviolet light radiation in the disinfectants solution has been effectively restored to its initial stage without any disease causing microorganisms.

The invention has been described with reference to the illustrated and presently preferred embodiment. It is not intended that the invention be unduly limited by this disclosure of the presently preferred embodiment. Instead, it is intended that the steps and their obvious equivalents, set forth in the claims, define the invention. It is also intended that the invention be construed as including all alterations and modifications insofar as they come within the scope of the appended claims or the equivalents thereof.

1. A method of disinfecting and sterilizing medical apparatus/instruments at low-temperature, which comprises:
immersing said apparatus/instruments in an aqueous liquid bath;
introducing aqueous liquid into said aqueous liquid bath and withdrawing aqueous liquid therefrom to effect washing of said apparatus/instruments and removal of desorbed microorganisms contaminates therefrom;
applying ultrasonic vibration at a predetermined frequency to said aqueous liquid bath and to said apparatus/instruments contained therein, said vibration being sufficient to effect a substantial desorption of said microorganisms contaminates from said apparatus/instruments;
introducing ozone into said aqueous liquid bath to oxidize said microorganisms contaminates; and
applying ultraviolet (UV) irradiation to said aqueous liquid bath and to said apparatus/instruments contained therein.

2. A method of disinfecting and sterilizing medical apparatus/instruments at low-temperature, which comprises:
immersing said apparatus/instruments in an aqueous liquid bath;
introducing aqueous liquid into said aqueous liquid bath and withdrawing aqueous liquid therefrom to effect washing of said apparatus/instruments and removal of desorbed microorganisms contaminates therefrom;
applying ultrasonic vibration at a predetermined frequency to said aqueous liquid bath and to said apparatus/instruments contained therein, said vibration being sufficient to effect a substantial desorption of said microorganisms contaminates from said apparatus/instruments;
introducing ozone into said aqueous liquid bath to oxidize said microorganisms contaminates.

3. A method of disinfecting and sterilizing medical apparatus/instruments at low-temperature, which comprises:
immersing said apparatus/instruments in an aqueous liquid bath;
introducing aqueous liquid into said aqueous liquid bath and withdrawing aqueous liquid therefrom to effect washing of said apparatus/instruments and removal of desorbed microorganisms contaminates therefrom;
applying ultrasonic vibration at a predetermined frequency to said aqueous liquid bath and to said apparatus/instruments contained therein, said vibration being sufficient to effect a substantial desorption of said microorganisms contaminates from said apparatus/instruments;
applying ultraviolet (UV) irradiation to said aqueous liquid bath and to said apparatus/instruments contained therein.

4. The method of claim 1, wherein said apparatus/instruments comprises at least one of endoscopes to be used in medical, dental, heart surgery and operations, and kidney dialysis instruments and all medical equipment that can be subject to low temperature disinfections or sterilization.

5. The method of claim 1, wherein said aqueous liquid bath is applied with at least one chemical reagent which is one of sterilants and disinfectants to contact with said immersed apparatus/instruments.

6. The method of claim 1, wherein said ultrasonic vibration is applied to said aqueous liquid bath by one of a reactor equipped with ultrasound generator and an ultrasound probe immersed into said aqueous liquid bath.

7. The method of claim 1, wherein said aqueous liquid bath can be maintained at a temperature from 20 degree C. to 100 degree C.

8. The method of claim 1, wherein said ozone is introduced into said aqueous liquid bath by passing one of air and pure oxygen through an ozone generator to form an ozone gas stream and introducing said ozone gas stream into said aqueous liquid bath.

9. The method of claim 1, wherein said ozone gas stream makes contact with the medical apparatus/instruments both on the surface and within the hollow lumen tubing along with the circulating liquid.

10. The method of claim 1, wherein said ultraviolet irradiation is applied to a reactor chamber with liquid stream circulating in and out of said apparatus/instruments.

11. A low-temperature sterilization, disinfections device, comprising:
a chamber, said chamber disposed to form an aqueous liquid bath therein;
ultrasonic vibration generator coupled to said chamber, said ultrasonic vibration generator adapted to supply ultrasonic vibration to said chamber;
ozone generator coupled to said chamber, said ozone generator adapted to supply ozone-containing gas to said chamber;
ultraviolet light source, adapted to expose said chamber with ultraviolet light.

12. A low-temperature sterilization, disinfections device, comprising:
a chamber, said chamber disposed to hold an aqueous liquid bath;
ultrasonic vibration generator coupled to said chamber, said ultrasonic vibration generator adapted to supply ultrasonic vibration to said chamber;
ozone generator coupled to said chamber, said ozone generator adapted to supply ozone-containing gas to said chamber;

13. A low-temperature sterilization, disinfections device, comprising:
a chamber, said chamber disposed to hold an aqueous liquid bath;
ultrasonic vibration generator coupled to said chamber, said ultrasonic vibration generator adapted to supply ultrasonic vibration to said chamber;
ultraviolet light source, adapted to expose said chamber with ultraviolet light.

14. The device of claim 11, further comprising:
chemical reagent supply, adapted to supply at least one chemical reagent to said chamber; and
said liquid bath contains from 1 to about 200,000 parts per million of at least one of sterilant and disinfections reagents.

15. The device of claim 11, wherein said ultrasonic vibration to said chamber is applied at a frequency from 1 to about 100 Mega-Hertz, said ultrasonic vibration having cavitations force being adapted to penetrate into small porous size and being sufficient to effect a substantial desorption and removal of microorganisms and all contaminates from said apparatus/instruments.

16. The device of claim 11, wherein said ultrasonic vibration is applied to said chamber by one of:
immersing a probe into said aqueous liquid bath to generate ultrasound vibration therein, or
equipping a reaction chamber with ultrasound generator to generate vibration therein.

17. The device of claim 11, wherein said aqueous liquid bath is maintained at a temperature from 20 degree C. to 100 degree C.

18. The device of claim 11, wherein said apparatus/instruments comprises at least one of medical instruments such as endoscopes to be used in medical, dental, heart surgery and operations, and kidney dialysis instruments and all medical equipment that can be subject to low temperature disinfections or sterilization.

19. The device of claim 11, further comprising:
introducing ozone into said aqueous liquid bath to oxidize said microorganisms and contaminates both in liquid phase and in gas phase of the potential air pockets of the hollow lumen tubing.

20. The device of claim 19, further comprising passing one of oxygen and atmosphere air through an ozone generator treatment thereby producing ozone, and introducing said ozone into said aqueous liquid bath.

21. The device of claim 11, wherein said ultraviolet irradiation has a wavelength from 10 to about 400 nm to said aqueous liquid bath.

22. The method of claim 2, wherein said apparatus/instruments comprises at least one of endoscopes to be used in medical, dental, heart surgery and operations, and kidney dialysis instruments and all medical equipment that can be subject to low temperature disinfections or sterilization.

23. The method of claim 3, wherein said apparatus/instruments comprises at least one of endoscopes to be used in medical, dental, heart surgery and operations, and kidney dialysis instruments and all medical equipment that can be subject to low temperature disinfections or sterilization.

24. The method of claim 2, wherein said aqueous liquid bath is applied with at least one chemical reagent which is one of sterilants and disinfectants to contact with said immersed apparatus/instruments.

25. The method of claim 3, wherein said aqueous liquid bath is applied with at least one chemical reagent which is one of sterilants and disinfectants to contact with said immersed apparatus/instruments.

26. The method of claim 2, wherein said ultrasonic vibration is applied to said aqueous liquid bath by one of a reactor equipped with ultrasound generator and an ultrasound probe immersed into said aqueous liquid bath.

27. The method of claim 3, wherein said ultrasonic vibration is applied to said aqueous liquid bath by one of a reactor equipped with ultrasound generator and an ultrasound probe immersed into said aqueous liquid bath.

28. The method of claim 2, wherein said aqueous liquid bath can be maintained at a temperature from 20 degree C. to 100 degree C.

29. The method of claim 3, wherein said aqueous liquid bath can be maintained at a temperature from 20 degree C. to 100 degree C.

30. The method of claim 2, wherein said ozone is introduced into said aqueous liquid bath by passing one of air and pure oxygen through an ozone generator to form an ozone gas stream and introducing said ozone gas stream into said aqueous liquid bath.

31. The method of claim 2, wherein said ozone gas stream makes contact with the medical apparatus/instruments both on the surface and within the hollow lumen tubing along with the circulating liquid.

32. The method of claim 3, wherein said ultraviolet irradiation is applied to a reactor chamber with liquid stream circulating in and out of said apparatus/instruments.

33. The device of claim 12, further comprising:
chemical reagent supply, adapted to supply at least one chemical reagent to said chamber; and
said liquid bath contains from 1 to about 200,000 parts per million of at least one of sterilant and disinfections reagents.

34. The device of claim 13, further comprising:
chemical reagent supply, adapted to supply at least one chemical reagent to said chamber; and
said liquid bath contains from I to about 200,000 parts per million of at least one of sterilant and disinfections reagents.

35. The device of claim 12, wherein said ultrasonic vibration to said chamber is applied at a frequency from 1 to about 100 Mega-Hertz, said ultrasonic vibration having cavitations force being adapted to penetrate into small porous size and being sufficient to effect a substantial desorption and removal of microorganisms and all contaminates from said apparatus/instruments.

36. The device of claim 13, wherein said ultrasonic vibration to said chamber is applied at a frequency from 1 to about 100 Mega-Hertz, said ultrasonic vibration having cavitations force being adapted to penetrate into small porous size and being sufficient to effect a substantial desorption and removal of microorganisms and all contaminates from said apparatus/instruments.

37. The device of claim 12, wherein said ultrasonic vibration is applied to said chamber by one of:
immersing a probe into said aqueous liquid bath to generate ultrasound vibration therein, or
equipping a reaction chamber with ultrasound generator to generate vibration therein.

38. The device of claim 13, wherein said ultrasonic vibration is applied to said chamber by one of:
immersing a probe into said aqueous liquid bath to generate ultrasound vibration therein, or
equipping a reaction chamber with ultrasound generator to generate vibration therein.

39. The device of claim 12, wherein said aqueous liquid bath is maintained at a temperature from 20 degree C. to 100 degree C.

40. The device of claim 13, wherein said aqueous liquid bath is maintained at a temperature from 20 degree C. to 100 degree C.

41. The device of claim 12, wherein said apparatus/instruments comprises at least one of medical instruments such as endoscopes to be used in medical, dental, heart surgery and operations, and kidney dialysis instruments and all medical equipment that can be subject to low temperature disinfections or sterilization.

42. The device of claim 13, wherein said apparatus/instruments comprises at least one of medical instruments such as endoscopes to be used in medical, dental, heart surgery and operations, and kidney dialysis instruments and all medical equipment that can be subject to low temperature disinfections or sterilization.

43. The device of claim 12, further comprising:
introducing ozone into said aqueous liquid bath to oxidize said microorganisms and contaminates both in liquid phase and in gas phase of the potential air pockets of the hollow lumen tubing.

44. The device of claim 13, wherein said ultraviolet irradiation has a wavelength from 10 to about 400 nm to said aqueous liquid bath.
